(19) United States
(12) Patent Application Publication
(10) Pub. No.: US 2003/0148952 A1
(43) Pub. Date: Aug. 7, 2003

(54) Methods and materials for the recruitment of endothelial cells

(76) Inventor: Timothy M. Crombleholme, Haverford, PA (US)

Correspondence Address: Kathleen D. Rigaut, P.h.D., J.D., Dann, Dorfman, Herrell And Skillman, Suite 720, 1601 Market Street, Philadelphia, PA, 19102, US

(21) Appl. No.: 10/273,880

(22) Filed: Oct. 18, 2002

Related U.S. Application Data
(60) Provisional application No. 60/330,317, filed on Oct. 18, 2001.
(60) Provisional application No. 60/376,916, filed on May 1, 2002.

(51) Int. Cl. A61K 48/00; A61K 38/24

(52) U.S. Cl. 514/12; 514/44; 424/93.2

(57) ABSTRACT

Methods are provided for recruiting endothelial precursor cells to wound sites

This application claims priority under 35 U.S.C. §119(e) to U.S. Provisional Application Nos. 60/330,317 filed Oct. 18, 2001 and 60/376,916 filed May 1, 2002.

Pursuant to 35 U.S.C. §202(c) it is acknowledged that the U.S. Government has certain rights in the invention described herein, which was made in part with funds from the National Institutes of Health, NIDDK Agency, Grant Number DK59242.

### FIELD OF THE INVENTION

This invention relates to the fields of wound healing and angiogenesis. More specifically, the invention provides materials and methods for the recruitment of endothelial cells to sites of vascular damage.

### BACKGROUND OF THE INVENTION

Several publications and patent documents are referenced in this application by numerals, for example, in order to more fully describe the state of the art to which this invention pertains. The disclosure of each of these publications and patent documents is incorporated by reference herein.

The endothelial lining of blood vessels is a highly complex, multi-functional cellular interface. Endothelial cells interact with both the blood and the underlying vessel wall components to maintain physiological homeostasis. The effects of endothelial injury have been studied in several experimental models designed to examine the development of biological mechanisms. After endothelial injury, the vessel wall loses its non-thrombogenic properties. One of the first events to occur is platelet adherence to the vessel surface, which is extensive over the first several days but diminishes over the following week (Steele et al., 1995, Circ. Res. 57:105-112). Platelets adhere to the subendothelium and secrete a variety of factors, including platelet derived growth factor (PDGF), which has been shown to be mitogenic for vascular smooth muscle cells (SMC) in vitro. Local release of this factor has been postulated to play a role in the genesis of intimal hyperplasia and atherosclerosis (Harker et al., 1976, J. Clin. Invest. 58, 731; Friedman et al., 1977, J. Clin. Invest. 60:1191). Additional substances released from platelets include heparitinase and platelet factor 4. The latter protein has high affinity for heparin and has been shown to penetrate into the vascular media after de-endothelialization. Macrophages, which are also a rich source of SMC mitogens, are frequently present in the injured area (Gimbrone, M. A. Jr., In: Jaffe, E. A., Editor, Biology of Endothelial Cells, Martinus Nijhoff Publishers, pp. 97-107 (1984). The final response of the injured arterial wall, independent of whether the injury is chemical, mechanical or biological, is characterized by proliferation of cells in the intima to form a fibro-musculo-elastic plaque (Hoff, 1970, Thromb. Haemostas. 40: 121.

The angiopoietin family comprises four members, all of which bind primarily to the TIE-2 receptor (1-3). It is not known whether they are also ligands for the TIE-1 receptor. Little is known about angiopoietin-3 and angiopoietin-4 which may represent diverging counterparts of the same gene locus in mouse and man (1-3). The angiopoietin (Ang) proteins were first discovered as ligands of the TIE-2 receptor (4,5). The TIE receptor tyrosine kinases were initially thought to be selectively expressed on vasculature and endothelium similar to VEGF receptors. It is now recognized that TIE receptors may be expressed on other cells such as those of the hematopoietic lineages and on fibroblasts. An angiopoietin-1 (Ang-1) or TIE-2 deficient embryo has a VEGF mediated primary vasculature that develops, but deficient embryos fail to remodel and stabilize the preformed vasculature and, therefore, die during embryogenesis (3,6,7). Transgenic overexpression of Ang-1 in the skin of mice leads to more numerous dermal capillaries and veins that are highly branched and characterized by increased diameters (7). Expression of exogenous VEGF leads to hyperpermeable and leaky blood vessels, whereas Ang-1 overexpression does not, even under inflammatory conditions (7). Ang-1 is constituently expressed in the adult, but Ang-2 is highly induced at sites of vascular remodeling in adults, such as the female reproductive tract (8-10). It is thought that Ang-2 leads to destabilization of existing vessels and connects to active vascular remodeling in the presence of VEGF (11).

### SUMMARY OF THE INVENTION

In accordance with the present invention, it has been appreciated that Ang-1 recruits endothelial cell precursors, which are bone marrow derived circulating angioblasts, to sites of vascular damage where they initiate angiogenesis and vasculogenesis. As described herein, Ang-1 mediated recruitment of endothelial precursor cells (EPCs) to sites of vascular damage may be used to advantage to initiate and promote healing at such sites, including, but not limited to wounds and damaged vascular linings. Indeed, Ang-1 mediated stimulation of tissue repair involves a reorganization of the wounded area which serves to create a cellular milieu conducive to tissue repair.

Also provided are methods wherein Ang-1 may be used to treat patients in which EPC recruitment is impaired or augmentation of EPC recruitment would be beneficial. Enhancement of EPC recruitment may be of particular utility in the treatment of diabetic patients who are impaired in processes related to wound healing. Of note in this regard, the present inventors have discovered that fewer EC precursors are recruited to wounds of diabetic mice relative to those of normal mice. Enhancement of EPC recruitment may also be used to advantage in the treatment of patients who have damaged vascular linings resulting from ischemia or a variety of surgical procedures. Patients who have undergone tissue or organ transplants, the viability of which depends on neovascularization, may also benefit from treatment using the methods of the present invention.

In accordance with the present invention, methods are provided for promoting recruitment of EPCs to a wound site to promote healing of the wound. Such methods comprise administering an Ang-1 expressing nucleic acid to a wound site to promote expression of an exogenous Ang-1 polypeptide, wherein the expression of the exogenous Ang-1 polypeptide in the wound promotes healing by recruiting EPCs to the wound site wherein these cells and their descendants promote healing and restoration of normal tissue architecture.

Gene transfer of an Ang-1 polypeptide or a fragment thereof having similar biologic effects may be achieved utilizing plasmid-, viral or non-viral-mediated gene transfer techniques to overexpress an Ang-1 polypeptide in a wound, and thereby recruit EPCs which promote wound healing. In an aspect of the present invention, nucleic acid sequences encoding Ang-1 may be administered to a patient in need thereof in a gene transfer vector to facilitate expression of Ang-1 polypeptide in the patient.

Viral vectors which may be used to advantage in the methods of the present invention include, but are not limited to, the group consisting of an adenoviral vector, an adeno-associated virus vector, a hybrid adeno-associated virus vector, a lentivirus vector, a pseudo-typed lentivirus vector, a herpes simplex virus (HSV) vector, a vaccinia virus vector, and a retroviral vector.

In one aspect, nucleic acid sequences encoding Ang-1 may be administered to a patient in an adenoviral vector to facilitate expression of Ang-1 protein. The present invention also encompasses the administration of Ang-1 protein to a patient to recruit EPCs to wounds, wherein such cells can promote healing.

Gene transfer of Ang-1, or a fragment of an Ang-1 gene, may be used in conjunction with a pharmaceutically acceptable carrier to create a permissive environment which promotes EPC recruitment, which accelerate the wound healing process.

Gene transfer of Ang-1, or a fragment of an Ang-1 gene, may be used in conjunction with a composition for promoting the healing of acute or chronic wounds, providing a permissive environment for healing to proceed wherein EPCs are actively recruited to the damaged tissue.

The methods of the present invention may be used to treat any patient with chronic non-healing wounds, peripheral ischemia, or myocardial ischemia, particularly those patients who are deficient for Ang-1 expression and/or activity. The methods of the present invention may be used to advantage to treat a variety of conditions characterized by ischemic events, such as, but not limited to cerebrovascular ischemia, subarachnoid hemorrhage, myocardial infarct, and those ischemic events resulting from surgery and trauma. In a particularly preferred embodiment, patients with diabetes may be treated therapeutically with Ang-1 to effect improvements in physiological processes related to EC recruitment, such as wound healing, diabetic neuropathy, angiogenesis, and ischemia. Ang-1 mediated recruitment of EPCs may also be used to advantage to promote engraftment of a transplanted tissue or organ in a recipient.

Also provided is a method comprising administering a therapeutically effective amount of an Ang-1 molecule in combination with therapeutically effective amounts of an insulin-like growth factor 1 molecule and a platelet derived growth factor B molecule, which act synergistically to recruit endothelial precursor cells to a wound site.

It is therefore an object of this invention to provide methods to recruit EPCs, wherein such recruitment enhances angiogenesis, wound healing, and the maintenance of organ transplants.

The invention will be even more apparent from the following examples, as detailed in the figures and description below.

### BRIEF DESCRIPTION OF THE DRAWINGS

FIG. 1 shows histograms of (A) the epithelial gap and (B) the capillary density in wounds treated with the indicated agents at 1, 3, 7, and 14 days post-wounding.

FIG. 2 shows a histogram of the number of endothelial precursor cells per high power field (EPCs/HPF) in normal and diabetic mouse wounds.

FIG. 3 shows a histogram of the number of EPCs/HPF in wounds treated with the indicated growth factors.

FIG. 4 shows a histogram comparing the number of EPCs/HPF and capillaries/HPF in wounds treated with the indicated growth factors.

### DETAILED DESCRIPTION OF THE INVENTION

The present inventors have discovered that EPC recruitment is promoted by the administration of Ang-1. Hence, Ang-1 molecules may be used to promote wound healing, repair of damaged vascular linings, and neo-vascularization of organ transplants, including artificial organ transplants. The present invention is, therefore, directed to methods for enhancing EPC recruitment to promote wound healing, repair of damaged vascular linings, and enhance organ transplantation in a patient in need thereof. In addition, the methods of the present invention may be used to enhance skin grafting and accelerate endothelial cell coverage of vascular grafts in order to prevent graft failure due to reocclusion.

Of note, atherosclerotic vascular disease remains the leading cause of death among Americans. As medical science has become more sophisticated, invasive vascular procedures are being applied to obstructed vessels in the absence of effective preventive or therapeutic drug modalities. Arterial endarterectomy and percutaneous balloon dilatation of vessels, for example, are routinely used to treat pathologic stenosis. Although these and other procedures are often successful, a common complication after these procedures is the occurrence of vessel wall abnormalities. These abnormalities include recurrent stenosis due to atherosclerosis, smooth muscle proliferation, and loss of vessel wall integrity as a result of fibrosis and thrombosis of the vessel. Injury to and/or removal of endothelial cells lining the blood vessels are common features inherent to vascular procedures, and current studies suggest that spontaneous re-endothelialization of these injuries may occur slowly, partially, or not at all.

It is evident that endothelial cells play a key role in the etiology of blood vessel dysfunction. Restoration of intact endothelium immediately following injury is likely to moderate the deleterious effects which result from the injury. Therefore, it is an object of the present invention to provide a method for re-endothelializing the linings of vascular passages that have been substantially denuded of endothelial cells. Patients who may benefit from the methods of the present invention include those who have been subjected to procedures that damage the endothelial cell linings of the vascular passages, e.g., percutaneous transluminal angioplasty.

The present invention is directed to a method for promoting the recruitment of endothelial precursor cells in a subject comprising the administration of Ang-1 molecules in a therapeutically effective amount. Ang-1 molecules of the present invention which are suitable for administration to a patient may be Ang-1 polypeptides or expression vectors comprising nucleic acid sequences encoding Ang-1 protein or a functional fragment thereof. Exemplary nucleic acid sequences encoding human Ang-1 and amino acid sequences of human Ang-1 are disclosed in U.S. Pat. No. 5,521,073, the entire contents of which is incorporated herein by reference. The invention is not intended, however, to be limited to Ang-1 derived from humans. Accordingly, Ang-1 derived from any compatible animal system may be used in the methods of the present invention.

In one embodiment of the present invention, nucleic acid sequences encoding Ang-1 may be administered to a patient in an expression vector. Vectors which may be used to advantage to express Ang-1 or derivatives thereof at wound sites include, but are not limited to, plasmid vectors and viral vectors. Such expression vectors are known to those of skill in the art and described hereinbelow. In a preferred embodiment, a viral vector comprising a nucleic acid sequence encoding Ang-1, or a functional fragment or derivative thereof, may be used according to the methods of the present invention. Viral vectors of utility for the methods of the present invention include, but are not limited to, adenoviral vectors, adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], HSV vectors, vaccinia virus vectors, and retroviral vectors. Such viral vectors are known to those of skill in the art and described hereinbelow.

Ang-1 and expression vectors thereof may be administered topically, intravenously, intramuscularly, intradermally, subcutaneously or intraperitoneally. Ang-1 polypeptide expression vectors may be administered alone, or in conjunction with expression vectors encoding molecules that confer improved healing. Expression vectors encoding Ang-1 may be administered alone, or in combination with other expression vectors, to a tissue in a biologically compatible or pharmaceutically acceptable composition.

Ang-1 and expression vectors thereof may be administered to promote recruitment of EC precursors, which initiate angiogenesis and thereby enhance wound healing and re-vascularization or neo-vascularization of organ transplants. Ang-1 may be administered either alone or in combination with other cytokines (e.g., IL-10), including, for example, growth factors. Ang-1 mediated recruitment of EPCs may be enhanced by administering Ang-1 in combination with a growth factor or a second adenoviral vector encoding such a growth factor. Such growth factors may be selected from the group consisting of insulin-like growth factor 1 (IGF1), platelet derived growth factor B (PDGF-B), transforming growth factor alpha (TGF-alpha), and fibroblast growth factor (FGF).

### I. Definitions

Various terms relating to the biological molecules of the present invention are used hereinabove and also throughout the specification and claims.

With reference to nucleic acids of the invention, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

The term “promoter region” refers to the transcriptional regulatory regions of a gene, which may be found at the 5′ or 3′ side of the coding region, or within the coding region, or within introns.

The term “vector” refers to a small carrier DNA molecule into which a DNA sequence can be inserted for introduction into a host cell where it will be replicated. An “expression vector” is a specialized vector that contains a gene with the necessary regulatory regions needed for expression in a host cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide or amino acid means a sequence having the properties of a given SEQ ID NO:. For example, when used with reference to an amino acid sequence, the phrase includes the sequence per se and molecular modifications that would not affect the basic and novel characteristics of the sequence.

With respect to antibodies of the invention, the term “immunologically specific” refers to antibodies that bind to one or more epitopes of a protein of interest (e.g., Ang-1), but which do not substantially recognize and bind other molecules in a sample containing a mixed population of antigenic biological molecules.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method used. For example, for diagnostic applications, depending on the complexity of the target sequence, the oligonucleotide probe typically contains 15-25 or more nucleotides, although it may contain fewer nucleotides.

The probes herein are selected to be “substantially” complementary to different strands of a particular target nucleic acid sequence. This means that the probes must be sufficiently complementary so as to be able to “specifically hybridize” or anneal with their respective target strands under a set of predetermined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to act functionally as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product.

The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “percent identical” is used herein with reference to comparisons among nucleic acid or amino acid sequences. Nucleic acid and amino acid sequences are often compared using computer programs that align sequences of nucleic or amino acids thus defining the differences between the two. For purposes of this invention comparisons of nucleic acid sequences are performed using the GCG Wisconsin Package version 9.1, available from the Genetics Computer Group in Madison, Wis. For convenience, the default parameters (gap creation penalty=12, gap extension penalty=4) specified by that program are intended for use herein to compare sequence identity. Alternately, the Blastn 2.0 program provided by the National Center for Biotechnology Information (at http://www.ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences.

The present invention also includes active portions, fragments, and derivatives of an Ang-1 polypeptide of the invention. An “active portion” of an Ang-1 polypeptide means a peptide which is less than a full length Ang-1 polypeptide, but which retains its essential biological activity, e.g., recruitment of endothelial precursor cells.

A “fragment” of an Ang-1 polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids.

A “derivative” of an Ang-1 polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, without fundamentally altering the essential activity of the wildtype Ang-1 polypeptide.

Conservative amino acid substitutions refer to the interchangeability of residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Preferred conservative amino acid substitution groups are: valine-leucine-isoleucine, phenylalanine-tyrosine, lysine-arginine, alanine-valine, and asparagine-glutamine.

As mentioned above, an Ang-1 polypeptide or protein of the invention includes any analogue, fragment, derivative or mutant which is derived from Ang-1 and which retains at least one property or other characteristic of Ang-1. Different “variants” of Ang-1 exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acid residues are substituted with conservative or non-conservative amino acids, (b) variants in which one or more amino acids are added to an Ang-1 polypeptide, (c) variants in which one or more amino acids include a substituent group, and (d) variants in which an Ang-1 sequence is fused with another peptide or polypeptide such as a fusion partner, a protein tag or other chemical moiety, that may confer useful properties to Ang-1, such as, for example, an epitope for an antibody, a polyhistidine sequence, a biotin moiety and the like.

Other Ang-1-like proteins of the invention include variants in which amino acid residues from one species are substituted for the corresponding residue in another species, either at conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post-translational modification forms result in derivatives of Ang-1 that retain any of the biological properties of Ang-1, they are included within the scope of this invention.

The term “functional” as used herein implies that the nucleic or amino acid sequence is functional for the recited assay or purpose.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples, which do not need to be listed here as such examples are known in the art. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

As used herein, the term “substantially denuded of endothelial cells” refers to a vascular passage in which the endothelial cell lining has been injured or removed to an extent likely to cause adverse side effects to the patient. Such injuries can be classified (in order of increasing severity) as level I, II or III injuries: level I injury resulting in exposure of principally basement membrane, level II injury resulting in exposure of primarily sub-basement membrane, interstitial collagen and internal elastic lamina, and level III injury resulting in exposure of deeper layers including the media and smooth muscle cells in areas of internal elastic lamina fracture.

As used herein, the term “angiogenesis” refers to the formation of blood vessels. Specifically, angiogenesis is a multistep process in which endothelial cells focally degrade and invade the underlying basement membrane, migrate through interstitial stroma toward an angiogenic stimulus, proliferate proximal to the migrating tip, organize into blood vessels, and reattach to newly synthesized basement membrane (see Folkman et al., 1985, Adv. Cancer Res. 43:175-203). These processes are controlled by soluble factors, cell-cell interactions, and the extracellular matrix (see Ingber et al., 1985, Cell 58:803-805).

As used herein, “EPC recruitment” refers to a condition(s) wherein endothelial precursor cells are attracted to a localized region. As used herein, the term generally refers to attracting endothelial precursor cells to a wound site wherein such cells can participate in reorganization of a wound site such that wound healing is stimulated.

As used herein, “neo-vascularization” or “re-vascularization” refer to formation of or re-establishment of vascular structures in a tissue or organ such that normal blood flow is restored to the tissue or organ.

### II. Preparation of Ang-1-Encoding Nucleic Acid Molecules and Ang-1 Polypeptides

A. Nucleic Acid Molecules

Nucleic acid molecules encoding an Ang-1 polypeptide of the invention may be prepared by two general methods: (1) synthesis from appropriate nucleotide triphosphates, or (2) isolation from biological sources. The availability of nucleotide sequence information, such as a full length nucleic acid sequence having SEQ ID NO: 1, enables preparation of isolated nucleic acid molecules of the invention by oligonucleotide synthesis. Alternatively, nucleic acid sequences encoding an Ang-1 polypeptide may be isolated from appropriate biological sources using standard protocols. Both methods utilize protocols well known in the art.

In a preferred embodiment, an Ang-1 cDNA clone may be isolated from a cDNA expression library of human or mouse origin. In an alternative embodiment, a genomic clone encoding Ang-1 may be isolated utilizing the Ang-1 polypeptide-encoding cDNA or a fragment thereof as a probe. Sequence information for genomic and cDNA clones encoding human or murine Ang-1 may be obtained, for example, from the GenBank depository. The GenBank Accession Number for human Ang-1, for example, is U83508. Alternatively, nucleic and amino acid sequences encoding Ang-1 have been previously disclosed in U.S. Pat. Nos. 5,650,490 and 5,643,755, the entire contents of which are incorporated herein by reference.

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Genomic clones of the invention encoding an Ang-1 polypeptide may be maintained in lambda phage FIX II (Stratagene).

Ang-1 polypeptide-encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of the cDNA having SEQ ID NO: 1. Such oligonucleotides are useful as probes for detecting Ang-1 expression.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 75%, but often, more than 90%, of the nucleotides of the sequence match over the defined length of the nucleic acid sequence referred to using a specific SEQ ID NO:. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

Additionally, the term “substantially complementary” refers to sequences that may not match a target sequence perfectly, but are capable of hybridizing to the target sequence under appropriate conditions.

Thus, the coding sequence may be that shown in SEQ ID NO: 1 or it may be a mutant, variant, derivative or allele of this sequence. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in SEQ ID NO: 1 yet encode a polypeptide with the same amino acid sequence.

On the other hand, the encoded polypeptide may comprise an amino acid sequence which differs by one or more amino acid residues from the amino acid sequence shown in SEQ ID NO: 2. Nucleic acid encoding a polypeptide which is an amino acid sequence mutant, variant, derivative or allele of the sequence shown in SEQ ID NO: 1 is further provided by the present invention. Nucleic acid encoding such a polypeptide may show greater than 60% homology with the coding sequence shown in SEQ ID NO: 1, greater than about 70% homology, greater than about 80% homology, greater than about 90% homology or greater than about 95% homology.

Oligonucleotide probes or primers, as well as the full-length sequence (and mutants, alleles, variants, and derivatives) are useful for identifying variants of an Ang-1 polypeptide having novel properties such as an enhanced ability to recruit endothelial precursor cells. The conditions of the hybridization can be controlled to minimize non-specific binding, and preferably stringent to moderately stringent hybridization conditions are used. The skilled person is readily able to design such probes, label them and devise suitable conditions for hybridization reactions, assisted by textbooks such as Sambrook et al (1989) and Ausubel et al (1992).

In some preferred embodiments, oligonucleotides according to the present invention that are fragments of the sequence shown in SEQ ID NO: 1 or any allele associated with an ability to promote endothelial precursor cell recruitment, are at least about 10 nucleotides in length, more preferably at least 15 nucleotides in length, more preferably at least about 20 nucleotides in length.

B. Proteins

A full-length Ang-1 polypeptide of the present invention may be prepared in a variety of ways, according to known methods. The protein may be purified from appropriate sources, e.g., transformed bacterial or animal cultured cells or tissues which express Ang-1, by immunoaffinity purification. However, this is not a preferred method due to the low levels of protein likely to be present in a given cell type at any time.

The availability of nucleic acid molecules encoding an Ang-1 polypeptide enables production of Ang-1 using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of Ang-1 may be produced by expression in a suitable prokaryotic or eukaryotic system. For example, part or all of a DNA molecule, such as a nucleic acid sequence having SEQ ID NO: 1 may be inserted into a plasmid vector adapted for expression in a bacterial cell, such as E. coli. Alternatively, in a preferred embodiment, tagged fusion proteins comprising Ang-1 can be generated. Such Ang-1-tagged fusion proteins are encoded by part or all of a DNA molecule, such as the nucleic acid sequence having SEQ ID NO: 1, ligated in the correct codon frame to a nucleotide sequence encoding a portion or all of a desired polypeptide tag which is inserted into a plasmid vector adapted for expression in a bacterial cell, such as E. coli, or a eukaryotic cell, such as, but not limited to, yeast and mammalian cells. Vectors such as those described above comprise the regulatory elements necessary for expression of the DNA in the host cell (e.g. E. coli) positioned in such a manner as to permit expression of the DNA in the host cell. Such regulatory elements required for expression include promoter sequences, transcription initiation sequences and, optionally, enhancer sequences.

Ang-1 and fusion proteins thereof, produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope, GST or the hemagglutinin epitope. Such methods are commonly used by skilled practitioners.

Ang-1 and fusion proteins thereof, prepared by the aforementioned methods, may be analyzed according to standard procedures. For example, such proteins may be subjected to amino acid sequence analysis, according to known methods.

As discussed above, a convenient way of producing a polypeptide according to the present invention is to express nucleic acid encoding it, by use of the nucleic acid in an expression system. A variety of expression systems of utility for the methods of the present invention are well known to those of skill in the art.

Accordingly, the present invention also encompasses a method of making a polypeptide, the method including expression from nucleic acid encoding the polypeptide (generally nucleic acid). This may conveniently be achieved by culturing a host cell, containing such a vector, under appropriate conditions which cause or allow production of the polypeptide. Polypeptides may also be produced in in vitro systems, such as reticulocyte lysates.

The use of polypeptides which are amino acid sequence variants, alleles, derivatives or mutants are also encompassed by the present invention. A polypeptide which is a variant, allele, derivative, or mutant may have an amino acid sequence that differs from that given in SEQ ID NO: 2 by one or more of addition, substitution, deletion and insertion of one or more amino acids. Preferred such polypeptides exhibit Ang-1 activity, as defined herein, including the ability to recruit endothelial precursor cells.

A polypeptide which is an amino acid sequence variant, allele, derivative or mutant of the amino acid sequence shown in SEQ ID NO: 2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. Particular amino acid sequence variants may differ from that shown in SEQ ID NO: 2 by insertion, addition, substitution or deletion of 1 amino acid, 2, 3, 4, 5-10, 10-20, 20-30, 30-40, 40-50, 50-100, 100-150, or more than 150 amino acids.

Polyclonal and monoclonal antibodies directed toward Ang-1 are commercially available (e.g., Research Diagnostics, Inc. or Abcam). According to the methods of the present invention, polyclonal or monoclonal antibodies that immunospecifically interact with Ang-1 can be utilized for identifying and purifying Ang-1. For example, antibodies may be utilized for affinity separation of proteins with which they immunospecifically interact. Antibodies may also be used to immunoprecipitate proteins from a sample containing a mixture of proteins and other biological molecules. Methods for making and using monoclonal and polyclonal antibodies are provided in Harlow and Lane (1988) Antibodies A Laboratory Manual, Cold Spring Harbor Laboratory Press. Other uses of anti-Ang-1 polypeptide antibodies are described below.

Antibodies according to the present invention may be modified in a number of ways. Indeed the term “antibody” should be construed as covering any binding substance having a binding domain with the required specificity. Thus, the invention covers antibody fragments, derivatives, functional equivalents and homologues of antibodies, including synthetic molecules and molecules whose shape mimics that of an antibody enabling it to bind an antigen or epitope.

Exemplary antibody fragments, capable of binding an antigen or other binding partner, are Fab fragments consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

### III. Uses of Ang-1 Polypeptide-Encoding Nucleic Acids and Protein

Ang-1 nucleic acids and polypeptides, according to this invention, may be used, for example, as therapeutic and/or prophylactic agents which promote endothelial cell recruitment. The present inventors have discovered that administration of Ang-1 molecules, either alone or in combination with other agents, serves to recruit endothelial precursor cells. Active recruitment of EPCs promotes wound healing processes in that EPCs and their cellular descendants serve to reorganize damaged tissue in a manner conducive to healing and promotion of angiogenesis.

A. Ang-1-Encoding Nucleic Acids

Ang-1 polypeptide-encoding nucleic acids may be used for a variety of purposes in accordance with the present invention. Ang-1 polypeptide-encoding DNA, RNA, or fragments thereof may be used as probes to detect the presence of and/or expression of endogenously or exogenously expressed Ang-1. Methods in which Ang-1 polypeptide-encoding nucleic acids may be utilized as probes for such assays include, but are not limited to: (1) in situ hybridization; (2) northern hybridization; and (3) assorted amplification reactions such as polymerase chain reactions (PCR).

In a preferred embodiment of the invention, an expression vector comprising nucleic acid sequences encoding Ang-1, or a functional fragment thereof, may be administered to a wound site. The resultant expression of Ang-1, or a functional fragment thereof, serves to recruit EPCs to the wound site wherein the EPCs and cells differentiated therefrom contribute to the healing process. Methods for administering Ang-1 molecules have been previously disclosed in U.S. Pat. No. 6,057,435 and International Application Number PCT/US95/12935, the entire contents of which are incorporated herein by reference. Expression vectors comprising Ang-1 nucleic acid sequences may be administered alone, or in combination with expression vectors comprising nucleic acid sequences encoding other molecules. According to the present invention, the expression vectors or combinations thereof may be administered to wound sites either alone or in a pharmaceutically acceptable or biologically compatible composition.

Expression vectors comprising Ang-1 nucleic acid sequences may be administered alone, or in combination with other effector molecules or expression vectors comprising nucleic acid sequences encoding such effector molecules. An expression vector comprising a nucleic acid sequence encoding, for example, IGF-1 or PDGF-B may be administered in conjunction with an expression vector comprising a nucleic acid sequence encoding an Ang-1 polypeptide to recruit EPCs which serve to promote wound healing.

The present inventors have discovered that, when expressed in combination, Ang-1, IGF-1, and PDGF-B act synergistically to recruit EPCs, which in turn promote wound healing. This novel finding provides methods for therapeutic regimens directed to combined administration of expression vectors encoding Ang-1, IGF-1, or PDGF-B to wound sites to promote healing of the injured tissue.

According to the present invention, the expression vectors or combinations thereof may be administered to wound sites either alone or in a pharmaceutically acceptable or biologically compatible composition.

In a preferred embodiment of the invention, an expression vector comprising nucleic acid sequences encoding Ang-1, or a functional fragment or derivative thereof, is a viral expression vector. Viral vectors which may be used to advantage in the methods of the present invention include, but are not limited to, the group consisting of an adenoviral vector, an adeno-associated virus vector, a hybrid adeno-associated virus vector, a lentivirus vector, a pseudo-typed lentivirus vector, a herpes simplex virus vector, a vaccinia virus vector, and a retroviral vector.

In one embodiment of the present invention, methods are provided for the administration of an adenoviral vector comprising nucleic acid sequences encoding Ang-1, or a functional fragment thereof. As described herein, expression of Ang-1 or functional fragments thereof, serves to recruit EPCs to a site of administration. Recruitment of EPCs to wound sites or grafted tissue, for example, promotes healing and revascularization of damaged tissue in need of repair. The adenoviral vector may be, for example, a type 5 with E1 deleted which contains an expression cassette with Ang-1 nucleic acid sequences under the control of a cytomegalovirus promoter. Preferably, the Ang-1 nucleic acid sequences are of human origin. The methods of the present invention are not, however, limited to the use of human Ang-1 polynucleotides and polypeptides, but rather encompass Ang-1 polynucleotides and polypeptides derived from compatible species.

Recombinant adenoviral vectors have found broad utility for a variety of gene therapy applications. Their utility for such applications is due largely to the high efficiency of in vivo gene transfer achieved in a variety of organ contexts.

Adenoviral vector systems are of particular utility in the methods of the present invention because they provide several unique features, including, but not limited to: i) the ability to infect all human skin cells at more than 95% efficiency, making lengthy selection periods unnecessary; ii) the ability to remain episomal and rarely integrate into the human genome; and iii) the generation of replication defective adenoviruses (such as, e.g., the dl7001 adenoviral vector), from which the E1 gene region (the transforming region) and the E3 gene region (the immune modulatory region) have, for example, been deleted;(iv) the expression of viral or foreign genes from an adenovirus genome does not require a replicating cell; (v) there is no association of adenovirus infection with human malignancy; and (vi) attenuated strains have been developed and used safely in humans as vectors for live vaccines. The high infection efficiency achieved with adenoviral vectors is not generally observed using other gene transfer techniques. Moreover, the recombinant adenoviruses of the present invention are non-lytic and do not induce apparent phenotypic changes in infected cells. Maintenance of an adenoviral expression vector in an episomal state is advantageous because the chance of integration-mediated mutation in the host chromosome is minimal and the expression time of encoded proteins is finite. Adenovirus-mediated gene expression in keratinocytes or fibroblasts, for example, remains stable in vitro for at least 2 to 6 weeks, depending on the rate of cellular proliferation. Furthermore, gene expression in human skin grafted to SCID mice lasts for at least 2 weeks. As described above, the limited duration of high level transgene expression is sufficient to promote EPC recruitment.

In one embodiment, an adenoviral vector of the present invention is a type 5 adenovirus, with E1 deleted, which comprises Ang-1 nucleic acid sequences under the control of a cytomegalovirus promoter.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for the expression of Ang-1 or functional fragments thereof.

Recombinant adenoviral vectors have found broad utility for a variety of gene therapy applications. Their utility for such applications is due largely to the high efficiency of in vivo gene transfer achieved in a variety of organ contexts.

Adenoviral Mediated Gene Therapy

Adenoviral particles may be used to advantage as vehicles for adequate gene delivery. Such virions possess a number of desirable features for such applications, including: structural features related to being a double stranded DNA nonenveloped virus and biological features such as a tropism for the human respiratory system and gastrointestinal tract. Moreover, adenoviruses are known to infect a wide variety of cell types in vivo and in vitro by receptor-mediated endocytosis. Attesting to the overall safety of adenoviral vectors, infection with adenovirus leads to a minimal disease state in humans comprising mild flu-like symptoms.

Improved adenoviral vectors and methods for producing these vectors have been described in detail in a number of references, patents, and patent applications, including: Mitani and Kubo (2002, Curr Gene Ther. 2(2):135-44); Olmsted-Davis et al. (2002, Hum Gene Ther. 13(11):1337-47); Reynolds et al. (2001, Nat Biotechnol. 19(9):838-42); U.S. Pat. Nos. 5,998,205 (wherein tumor-specific replicating vectors comprising multiple DNA copies are provided); 6,228,646 (wherein helper-free, totally defective adenovirus vectors are described); 6,093,699 (wherein vectors and methods for gene therapy are provided); 6,100,242 (wherein a transgene-inserted replication defective adenovirus vector was used effectively in in vivo gene therapy of peripheral vascular disease and heart disease); and International Patent Application Nos. WO 94/17810 and WO 94/23744.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992). The incorporation of tissue specific regulatory elements in the expression constructs of the present invention provides for at least partial tissue tropism for the expression of Ang-1 or functional fragments thereof. For example, an E1 deleted type 5 adenoviral vector comprising nucleic acid sequences encoding Ang-1 under the control of a cytomegalovirus (CMV) promoter may be used to advantage in the methods of the present invention.

B. Ang-1 Polypeptides and Antibodies

Ang-1 polypeptides may be used for a variety of purposes in accordance with the present invention. In a preferred embodiment of the present invention, Ang-1 polypeptides and functional fragments and derivatives thereof may be administered to recruit endothelial precursor cells to a patient's wound. Ang-1 and functional derivatives thereof may be administered alone or in a composition so as to deliver a therapeutically effective amount of an Ang-1 polypeptide to a wound. An appropriate composition in which to deliver Ang-1 polypeptides may be determined by a medical practitioner upon consideration of a variety of physiological variables, including, but not limited to, the patient's condition and the wound site. A variety of compositions well suited for different applications and routes of administration are well known in the art and described hereinbelow.

It will be apparent to those of skill in the art that an Ang-1 molecule, or a derivative or fragment thereof, may be used either alone or in conjunction with other therapeutic agent(s) used for treating wounds. Such agents include, but are not limited to, IGF-1, IL-10, PDGF-B, and/or antibiotics.

From the foregoing discussion, it can be seen that Ang-1 polypeptide-encoding nucleic acids, Ang-1 polypeptide expressing vectors, cells comprising Ang-1 polypeptide expressing vectors, and Ang-1 polypeptides may be used in the treatment of wounds to promote EPC recruitment which serve to promote healing of the damaged tissue.

C. Pharmaceutical Compositions

The expression vectors of the present invention may be incorporated into pharmaceutical compositions that may be delivered to a subject, so as to allow production of a biologically active protein (e.g., an Ang-1 polypeptide or functional fragment or derivative thereof). In a particular embodiment of the present invention, pharmaceutical compositions comprising sufficient genetic material to enable a recipient to produce a therapeutically effective amount of an Ang-1 polypeptide can promote angiogenesis and wound healing in the subject. The compositions may be administered alone or in combination with at least one other agent, such as a stabilizing compound, which may be administered in any sterile, biocompatible pharmaceutical carrier, including, but not limited to, saline, buffered saline, dextrose, and water. The compositions may be administered to a patient alone, or in combination with other agents, angiogenic modulators, drugs (e.g., antibiotics) or hormones. In preferred embodiments, the pharmaceutical compositions also contain a pharmaceutically acceptable excipient. Such excipients include any pharmaceutical agent that does not itself induce an immune response harmful to the individual receiving the composition, and which may be administered without undue toxicity. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., 18th Edition, Easton, Pa. [1990]).

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art. The pharmaceutical compositions of the present invention may be manufactured in any manner known in the art (e.g., by means of conventional mixing, dissolving, granulating, dragee-making, levigating, emulsifying, encapsulating, entrapping, or lyophilizing processes).

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

After pharmaceutical compositions have been prepared, they may be placed in an appropriate container and labeled for treatment. For administration of Ang-1-containing vectors, such labeling would include amount, frequency, and method of administration.

Pharmaceutical compositions suitable for use in the invention include compositions wherein the active ingredients are contained in an effective amount to achieve the intended therapeutic purpose. Determining a therapeutically effective dose is well within the capability of a skilled medical practitioner using the techniques provided in the present invention. Visual examination of a healing wound, for example, is a simple and preferred method for measuring the efficacy of Ang-1 mediated gene therapy, although other techniques known in the art may also be used. Restoration of normal blood flow to a damaged tissue or transplanted organ, for example, may readily be determined utilizing techniques involving labeled reagents which are detectable by sophisticated medical techniques [e.g., magnetic resonance imaging (MRI)]. The function of a transplanted organ (e.g., a liver transplant) may also be determined by measuring serum levels of specific metabolic/catabolic enzymes or their by-products.

Therapeutic doses will depend on, among other factors, the age and general condition of the subject, the severity of the wound, the size of the tissue transplant or graft, and the strength of the control sequences regulating the expression levels of an Ang-1 polypeptide. Thus, a therapeutically effective amount in humans will fall in a relatively broad range that may be determined by a medical practitioner based on the response of an individual patient to vector-based Ang-1 treatment. Guidelines pertaining to therapeutically effective amounts of Ang-1 have been previously addressed in U.S. Pat. Nos. 5,650,490; 5,972,338; 6,224,566; 6,262,333; and 6,262,334, the entire contents of which are incorporated herein by reference.

D. Administration

Expression vectors of the present invention comprising nucleic acid sequences encoding Ang-1, or functional fragments thereof, may be administered to a patient by a variety of means (see below) to achieve and maintain a prophylactically and/or therapeutically effective level of the Ang-1 polypeptide. One of skill in the art could readily determine specific protocols for using the Ang-1 encoding expression vectors of the present invention for the prophylactic and/or therapeutic treatment of a particular patient. Protocols for the generation of adenoviral vectors, for example, and administration to patients have been described in U.S. Pat. Nos. 5,998,205; 6,228,646; 6,093,699; 6,100,242; and International Patent Application Nos. WO 94/17810 and WO 94/23744., which are incorporated herein by reference in their entirety.

Ang-1 encoding viral expression vectors of the present invention may be administered to a patient by any means known. Direct delivery of the pharmaceutical compositions in vivo may generally be accomplished via injection using a conventional syringe, although other delivery methods such as convection-enhanced delivery are envisioned (See e.g., U.S. Pat. No. 5,720,720, incorporated herein by reference). In this regard, the compositions may be delivered subcutaneously, epidermally, intradermally, intrathecally, intraorbitally, intramucosally, intraperitoneally, intravenously, intraarterially, orally, intrahepatically or intramuscularly. Other modes of administration include oral and pulmonary administration, suppositories, and transdermal applications. In particularly preferred embodiments, the compositions may administered intravenously in an artery which provides blood flow to an organ in question. A clinician specializing in the treatment of patients who have received organ or tissue transplants may determine the optimal route for administration of the viral expression vectors comprising Ang-1 nucleic acid sequences based on a number of criteria, including, but not limited to: the condition of the patient and the purpose of the treatment (e.g., promotion of wound healing or vascularization of a transplanted organ or tissue graft).

In accordance with the present invention, adenoviral vectors comprising a nucleic acid sequence encoding an Ang-1 polypeptide may be administered to a wound at a dose range of 10⁵-10¹¹ plaque forming units (PFU). In a preferred embodiment, adenoviral vectors comprising a nucleic acid sequence encoding an Ang-1 polypeptide may be administered to a wound at a dose range of 10⁸-10¹⁰ PFU.

The present invention also encompasses AAV vectors comprising a nucleic acid sequence encoding an Ang-1 polypeptide, which may be administered to a wound at a dose range of 10⁶-10¹² PFU. In a preferred embodiment, AAV vectors comprising a nucleic acid sequence encoding an Ang-1 polypeptide may be administered to a wound at a dose range of 10⁸-10¹⁰ PFU.

Also provided are lentivirus or pseudo-typed lentivirus vectors comprising a nucleic acid sequence encoding an Ang-1 polypeptide, which may be administered to a wound at a dose range of 10⁷-10¹⁰ genome copies. In a preferred embodiment, lentivirus or pseudo-typed lentivirus vectors comprising a nucleic acid sequence encoding an Ang-1 polypeptide may be administered to a wound at a dose range of 10⁸-10¹⁰ genome copies.

In accordance with the present invention, HSV vectors comprising a nucleic acid sequence encoding an Ang-1 polypeptide may be administered to a wound at a dose range of 10⁶-10¹² PFU. In a preferred embodiment, HSV vectors comprising a nucleic acid sequence encoding an Ang-1 polypeptide may be administered to a wound at a dose range of 10⁷-10⁹ PFU.

Also encompassed are naked plasmid or expression vectors comprising a nucleic acid sequence encoding an Ang-1 polypeptide, which may be administered to a wound at a dose range of 5-20 μg.

The above protocols for administration of vectors comprising nucleic acid sequences encoding an Ang-1 polypeptide are based on an average wound area of approximately 1 cm². A skilled practitioner would appreciate that an appropriate dose of a vector encoding an Ang-1 polypeptide should be adjusted according to the application. The appropriate dose for a larger wound or organ transplant, for example, may be calculated based on the size of the area in need of treatment. A medical practitioner could readily determine the appropriate dose of administration based on the surface area of the wound relative to that of an average 1 cm² wound.

One skilled in the art will recognize that the methods and compositions described above are also applicable to a range of other treatment regimens known in the art. For example, the methods and compositions of the present invention are compatible with ex vivo therapy (e.g., where cells are removed from the body, incubated with the Ang-1 encoding adenoviral vectors and the treated cells are returned to the body).

Accordingly, Ang-1 encoding expression vectors or cells expressing such vectors may be administered to any tissue suitable for expression of Ang-1 polypeptides or fragments thereof.

In accordance with the present invention, Ang-1 encoding expression vectors or cells expressing such vectors may be administered to a tissue in need thereof, prophylactically (as part of a pre-treatment regimen), at the time of a procedure (such as a surgical procedure), or at presentation (after the injury has occurred).

### IV. Exemplary Applications for the Methods of the Invention

Treatment of Wounds and Transplants

The methods of the present invention may be used to particular advantage when performed in the context of organ transplantation. It is well known that the vascular endothelial cells of donor organs are damaged primarily during the cold preservation and reperfusion steps of organ transplantation procedures. The integrity of the vascular endothelium in transplants plays a critical role in the survival of the graft. Endothelial cells are central to the development of immune inflammatory processes, such as those involved in graft rejection. Moreover, endothelial cell death has been associated with vascular disease such as atherosclerosis. Despite intense research efforts to investigate the pathogenesis of endothelial cell damage during preservation and injury during ischemia-reperfusion, effective preventive measures and treatment remain elusive.

In the present invention, genetic modification of an organ for transplantation with an expression vector encoding the Ang-1 gene or a functional fragment thereof during the preservation step is disclosed. The expression of Ang-1 in a transplanted organ serves to improve the success rate of engraftment by recruiting EPCs from the transplant recipient to the transplanted organ. As described herein, recruitment of EPCs serves to promote vasculogenesis and/or angiogenesis and, thereby, accelerate and enhance the restoration of normal blood flow levels to a transplanted organ. The methods of the present invention may be used to advantage to genetically modify any organ or tissue intended for transplantion, including, but not limited to, autografts, allografts, and xenografts.

A therapeutically effective range of an Ang-1 polypeptide is approximately 1-30 μg for a wound of approximately 1 cm². A preferred therapeutically effective range of an Ang-1 polypeptide is between approximately 5-10 μg for a wound of approximately 1 cm². As described herein, the amount of Ang-1 polypeptide which constitutes a therapeutically effective amount is correlated with the size of the wound being treated. A therapeutically effective amount of Ang-1 encoded by an expression vector of the present invention varies, therefore, according to the surface area of the wound. A medical practitioner could readily determine the amount of an exogenous Ang-1 polypeptide that would provide a therapeutically effective amount for any wound, based on a calculation of the relative surface area of the wound as compared to that of the average 1 cm² wound.

Exemplary applications in which Ang-1 nucleic acids, Ang-1 polypeptide expressing vectors, Ang-1 polypeptides, or cells expressing Ang-1 may be used include treatment of acute or chronic wounds. Such treatment promotes active recruitment of EPCs to the damaged tissue wherein these cells and their progeny promote healing. The methods of the present invention may, for example, be used to treat any patient with chronic non-healing wounds, peripheral ischemia, or myocardial ischemia, particularly those patients who are deficient for Ang-1 expression and/or activity. In a particularly preferred embodiment, patients with diabetes may be treated therapeutically with Ang-1 to effect improvements in physiological processes related to EPC recruitment, such as wound healing, diabetic neuropathy, angiogenesis, and ischemia. Ang-1 mediated recruitment of EPCs may also be used to advantage to promote engraftment of a transplanted tissue or organ in a recipient.

In one embodiment of the present invention, a liver or portion thereof to be used for transplantation may be treated using the methods described herein. Of note, injury to the liver resulting from ischemia/reperfusion is a major consideration in numerous clinical settings, including, but not limited to, hepatic surgery, liver transplantation, shock states, and thermal injury. Significant liver damage occurs during cold preservation, anaerobic rewarming, and reperfusion of a liver transplant prior to transfer to a recipient host. Strategies for protecting the liver from damage by ischemia/reperfusion are, therefore, essential. The methods of the present invention may be used to advantage to reduce the degree of ischemia/reperfusion injury to a liver transplant prior to, during, and after the transplantation procedure. Viral expression vector-mediated transfer of nucleic acid sequences encoding Ang-1, or a fragment thereof, into a liver transplant prior to transfer to a recipient and expression of Ang-1 polypeptides therein provides a biochemical homing signal for EPCs of the recipient to migrate to the liver transplant. Following migration to the liver transplant, the EPCs can then differentiate to endothelial cells and participate in re-vascularization and tissue repair of the graft.

Early graft function after liver transplantation is determined in large part by the quality of the donor organ at retrieval. Secondary influences such as those related to hypothermic preservation, flush, and reperfusion injury also contribute significantly as described hereinabove. These processes may also play a role in the frequency of rejection, the development of vascular and biliary complications, and overall graft survival. Cell death (or apoptosis) plays an important role in a wide variety of pathophysiological circumstances including organ preservation. According to the methods of the present invention, the administration of a recombinant viral expression vector encoding Ang-1 or a functional fragment thereof to a liver transplant prior to transplantation serves to reduce cell death by promoting vascularization of the graft and restoration of normal tissue architecture. In promoting vascularization, the methods of the present invention improve the short and long term viability of the graft by restoring normal levels of blood flow and the nutrients therein to the transplant. Restoration of blood circulation to the graft also ensures that cellular toxins, which accumulate normally and those generated during the transplantation process, are removed efficiently.

The present invention is also directed to a method of reducing ischemia/reperfusion injury in the liver of an individual in need of such treatment, comprising the step of contacting the liver or liver cells with a viral expression vector comprising an Ang-1 encoding nucleic acid sequence.

The present invention is also directed to a method of improving organ preservation, comprising the step of contacting the organ with a viral expression vector comprising an Ang-1 encoding nucleic acid sequence.

From the foregoing discussion, it can be seen that Ang-1 polypeptide-encoding nucleic acids, Ang-1 polypeptide expressing vectors, cells comprising Ang-1 polypeptide expressing vectors, and Ang-1 polypeptides may be used in the treatment of conditions in which endothelial precursor cell recruitment is beneficial to a patient.

The following examples are provided to illustrate certain embodiments of the invention. They are not intended to limit the invention in any way.

### Example I

As described herein, an adenoviral construct comprising the human angiopoietin-1 gene was introduced into the db/db mouse strain to assess the effect of angiopoietin-1 expression on wound healing. The db/db mouse strain was chosen for these studies because it is considered a model system for excisional wound healing. The over-expression of angiopoietin-1 in diabetic excisional wounds resulted in accelerated wound healing with a statistically significant enhanced rate of wound closure, increased capillary cell density, and a reduction in inflammatory cells.

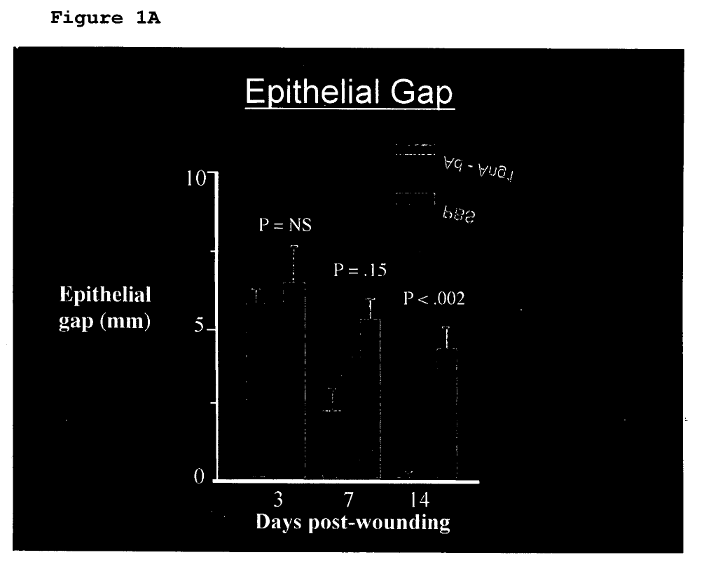

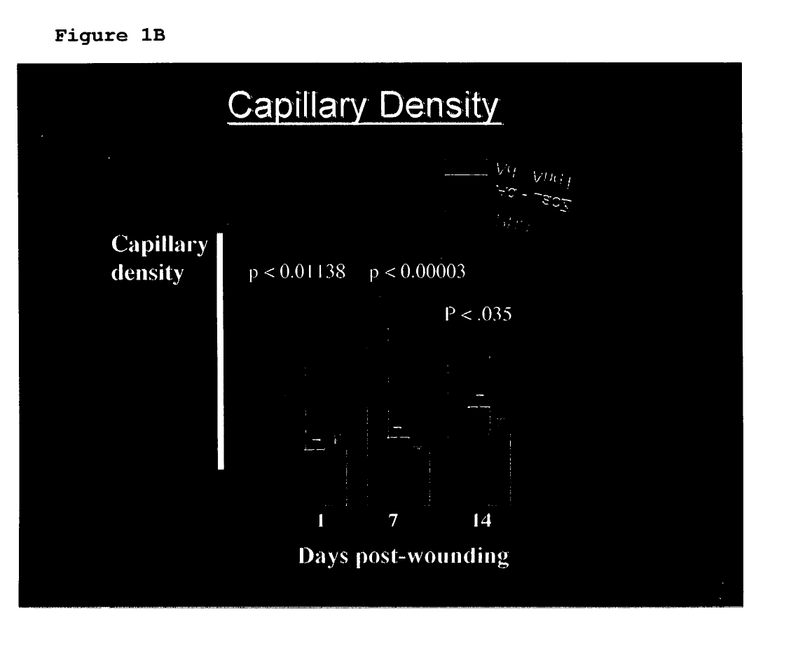

At three days post-wounding, the difference in the epithelial gap in wounds treated with PBS (phosphate buffered saline; media control), adenoviral vector comprising nucleic acid sequences encoding LacZ (Ad-LacZ), and Ad-Ang-1 was not significant. At seven days post-wounding, the epithelial gap of wounds treated with Ad-Ang-1 was significantly reduced as compared to those of wounds treated with media or vector control. At fourteen days post-wounding, the epithelial gap of wounds treated with Ad-Ang-1 was even more significantly reduced as compared to that of the media or vector control treated wounds. See FIG. 1A.

At one day post-wounding, the difference in the capillary density of wounds treated with PBS, Ad-LacZ, and Ad-Ang-1 was not significant. At seven days post-wounding, the capillary density of wounds treated with Ad-Ang-1 was significantly reduced as compared to the density of wounds treated with media or vector control. At fourteen days post-wounding, the capillary density of wounds treated with Ad-Ang-1 was even more significantly reduced as compared to that of the media and vector control treated wounds. See FIG. 1B.

Histologic examination of the rapidly healing wounds treated with Ad-Ang-1 (1×10⁸) revealed a unique cellular infiltrate, which was noted as early as three days after treatment. The number of infiltrating cells peaked at day 7 and was dramatically reduced at day 14 following administration of Ad-Ang-1. The infiltrating cells had eccentric pyknotic nuclei and abundant eosinophilic cytoplasm. In areas of granulation tissue formation, these cells were observed in palisades which exhibited subtle alterations in morphology. Some of the cells in the palisades had acquired a more endotheloid appearance characterized, in part, by a reduced cytoplasmic volume.

In order to characterize these cells, immunohistochemistry was performed using antibodies which recognize different antigens expressed by endothelial cells, as well as antigens expressed by endothelial precursor cells, including FLK-1 (VEGFR-2), MUC-18, PECAM, and TIE-2. Some of these markers, such as FLK-1 and TIE-2, may also be expressed by mature endothelial cells, whereas PECAM is not expressed on non-endothelial cells. MUC-18, however, is expressed by endothelial precursor cells and not by mature endothelial cells. The unique infiltrating cells were positive for FLK-1 (measured at day 7 post-wounding), TIE-2, and MUC-18, but had variable staining for PECAM, strongly suggesting that these cells were tissue endothelial progenitor cells.

In order to demonstrate that these unique cells were endothelial precursor cells, excisional wounds were harvested from db/db mice that had been treated with 1×10⁸ plaque forming units (pfu) of Ad-Ang-1 harvested at days 3, 7, and 14. These wounds were processed for RNA analysis and in situ hybridization to detect expression of the nuclear transcription factor GATA-2, which is expressed exclusively in endothelial precursor cells. In situ hybridizations were performed using anti-sense and sense (negative control) probes, the binding of which was detected utilizing a streptavidin and horseradish peroxidase coupled system. The in situ hybridization reaction using the control sense probe revealed no labeling, whereas the anti-sense GATA-2 probe hybridized specifically to those cells possessing the unique morphology characteristic of endothelial precursor cells. The data presented herein, therefore, provide the first demonstration of endothelial precursor cells in tissue prior to differentiation into mature endothelial cells.

It was not clear whether these endothelial precursor cells were derived from precursor cells resident in the tissue prior to wounding and treatment with Ad-Ang-1 or had been recruited from the circulation to the site of Ang-1 overexpression. Accordingly, a bone marrow transplant model was used to distinguish these two possibilities. Transgenic mice expressing green fluorescent protein (GFP) were used as bone marrow donors. The mice were euthanized and bone marrow was harvested from hind limbs in PBS. A FICOLL gradient was used to separate the low density mononuclear cells from the total cellular population, after which the mononuclear cells were washed and counted.

Syngeneic recipient mice underwent lethal irradiation with 828 cGy and six hours later were injected with low density mononuclear cells derived from donor bone marrow via the tail vein. After a four week recovery period to allow for engraftment, peripheral blood was sampled for analysis by fluorescence activated cell sorting (FACS) to determine the level of engraftment. If the level of donor engraftment was greater than 50%, the engrafted mouse was deemed ready to undergo wounding. Excisional wounds (10 millimeters) were created on the backs of these bone marrow transplanted mice and the wounds were either left untreated (negative control) or treated with 1×10⁸ pfu of Ad-Ang-1. The wounds were examined after seven days using a Wood's lamp to detect the presence of GFP expressing cells. Wounds treated with Ad-Ang-1 showed bright GFP positivity, whereas control wounds exhibited minimal levels of GFP positive cells.

Analysis of histological sections of the Ad-Ang-1 treated wounds revealed that the endothelial cells in the bed of the healed wound were brightly positive for GFP. In contrast, the minimal levels of GFP detected in the control wound were largely due to inflammatory cell infiltrate. These results demonstrate that the endothelial precursor cells in Ad-Ang-1 treated wounds were bone marrow derived. These findings are consistent with vasculogenesis playing a role as a source of new vessel formation and contributing to the appearance of mature endothelial cells found in these wounds.

These results were confirmed by performing a similar experiment in C57/BL6 female mice that had undergone lethal irradiation and bone marrow transplantation using GFP positive bone marrow cells derived from male donors. The bone marrow reconstituted female mice were excisionally wounded, treated with 1×10⁸ pfu of Ad-Ang-1, and harvested at seven days post-wounding. Analysis of histologic sections of wounds probed with a Y chromosome specific probe revealed that the cells infiltrating the wound were Y chromosome positive, thus demonstrating their origin as bone marrow cells derived from male donor mice.

Double label immunofluorescence experiments were performed on histologic sections of flank wounds of C57/BL6 mice which had undergone lethal irradiation and bone marrow reconstitution with bone marrow cells derived from GFP positive donors. Double labeling of such sections (harvested at day seven post-treatment) to detect GFP and the transcription factor GATA-2 revealed the presence of infiltrating cells in wounds treated with Ad-Ang-1 (1×10⁸ pfu) which were both bone marrow derived and endothelial precursor cells, as evidenced by the expression of GFP and GATA-2, respectively. Double label immunofluorescence of such sections to detect GFP and PECAM revealed the presence of infiltrating cells in wounds treated with Ad-Ang-1 (1×10⁸ pfu) which were mature endothelial cells of bone marrow origin, as evidenced by the expression of PECAM (a marker of mature endothelial cells) and GFP, respectively. These results demonstrate that infiltrating bone marrow derived endothelial precursor cells can differentiate into mature endothelial cells in treated wounds.

Intriguingly, at thirty days post-wounding and Ad-Ang-1 treatment, the number of GFP positive cells in wounds had decreased significantly. These data suggested that Ang-1 recruitment of EPCs might also initiate apoptotic pathways in recruited EPCs, thereby providing a means to down-regulate the wound repair process. As described herein, prior to apoptosis or programmed cell death, these cells are thought to reorganize the damaged tissue and stimulate repair thereof.

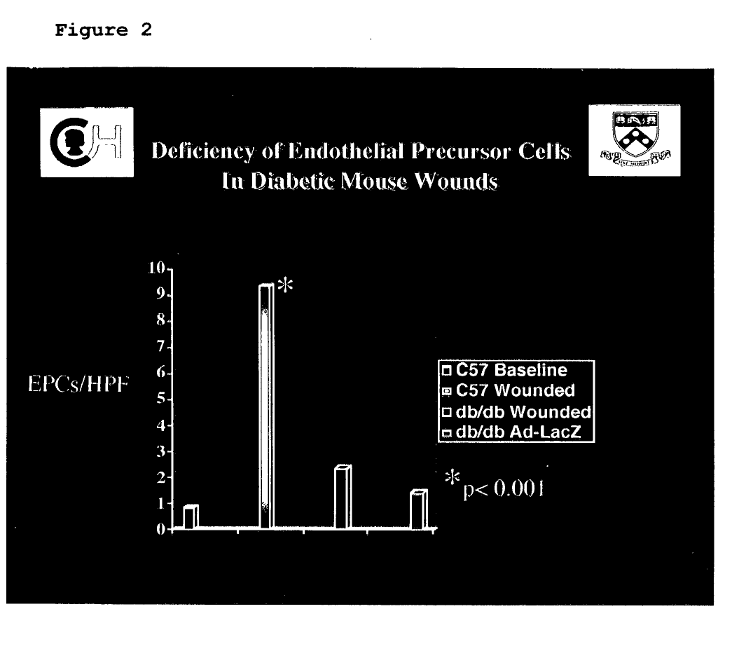

To investigate if diabetic mice were specifically deficient in their ability to recruit endothelial precursor cells to sites of neovascularization, the number of endothelial precursor cells/high power field (EPC/HPF) present in wounds of normal control C57 mice was compared with the number of EPCs in wounds of db/db mice. As shown in FIG. 2, there was a statistically significant reduction in the number of endothelial precursor cells recruited to the diabetic wounds as compared to the normal control wounds (as measured at day 7 post-wounding and Ad-Ang-1 administration). Results derived from other mouse model systems of diabetes, such as non-obese diabetic (NOD) mice and streptotozocin induced diabetic mice, also confirmed the above findings. Taken together, these data suggest that impaired ability to recruit EPCs to wound sites, as described herein for mouse models of diabetes, underlies the impaired wound healing and neovascularization observed in diabetic patients.

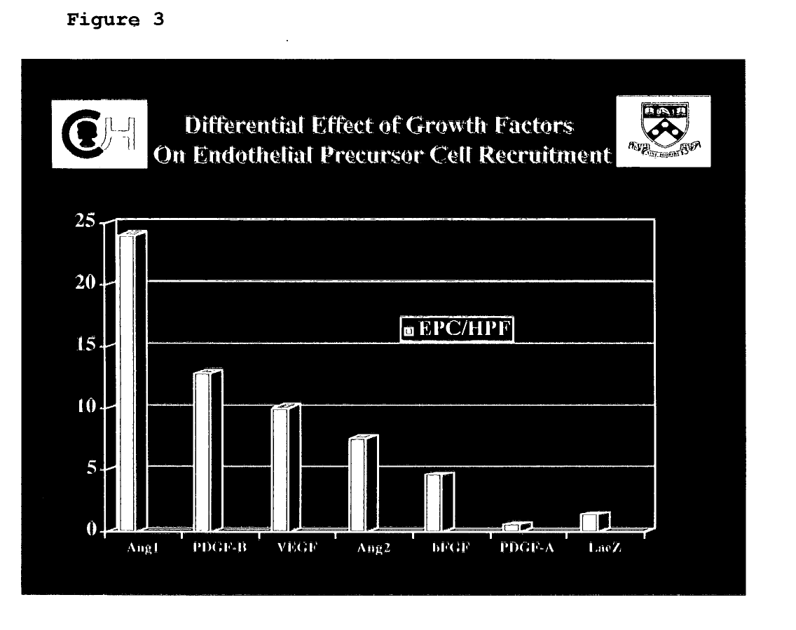

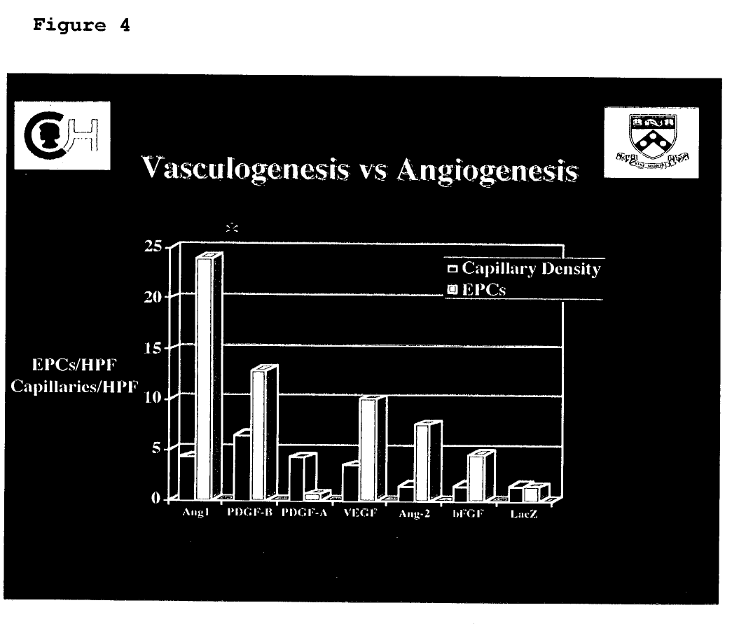

To provide a comparative analysis of angiogenic mediators, the ability of the cytokine Ang-1 to recruit endothelial precursor cells was compared to that of other known angiogeneic growth factors. The assessment was particularly significant with regard to the activity of VEGF, which is thought to mediate vasculogenesis in embryonic development. As shown in FIG. 3, Ang-1 proved to be a potent mediator of endothelial cell recruitment and was significantly more effective than any of the other angiogenic growth factors tested. Intriguingly, the ability of different growth factors to mediate endothelial cell recruitment was not consistent with their ability to mediate changes in capillary density. See FIG. 4. These results suggest that growth factors can differentially influence vasculogenic and angiogenic components of neovascularization.

Results presented herein demonstrate that Ang-1 is a potent mediator of EPC recruitment. Accordingly, the present inventors provide methods for the administration of Ang-1 to patients in need thereof to effect EPC recruitment to wound sites. Ang-1 may be used to advantage in therapeutic and/or prophylactic treatment of patients having disorders in which neovascularization is desirable. Such disorders or conditions include, but are not limited to, chronic non-healing diabetic wounds, peripheral vascular ischemia, myocardial ischemia, and diabetic neuropathy.

References

1. Sato et al. Nature 376: 70-74, 1995

2. Folkman et al. Cell 87: 1153-1155, 1996

3. Suriet et al. Cell 87: 1171-1180, 1996

4. Risau. Nature 386: 671-674, 1997

5. Risau and Flamme. Annu Rev Cell. Dev biol 11: 73-91, 1995

6. Dumont et al. Genes Dev 8: 1909-1994,

7. Sato et al. Proc Natl Acad Sci USA 90: 9355-9358, 1993

8. Maisonpierre et al. Science 277: 55-60, 1997

9. Thurston et al. Science 286: 2511-2514, 1999

10. Carmeliet. Curr Inter Cord Rep 1: 322-335, 1999

11. Maisonpierre et al. Oncogene 8: 1631-1637

What is claimed is:

1. A method for recruiting endothelial precursor cells to a wound site in a subject, comprising administering a therapeutically effective amount of an angiopoietin-1 molecule to effect recruitment of endothelial precursor cells.

2. The method as claimed in claim 1, wherein said angiopoietin-1 molecule is a nucleic acid sequence encoding an angiopoietin-1 polypeptide or a functional fragment thereof, wherein expression of said Ang-1 polypeptide or functional fragment thereof at said wound site promotes recruitment of endothelial precursor cells to said wound site.

3. The method as claimed in claim 2, wherein said nucleic acid sequence is included in an expression vector.

4. The method as claimed in claim 3, wherein said nucleic acid is delivered in an expression vector selected from the group consisting of a plasmid vector and a viral vector.

5. The method as claimed in claim 4, wherein said viral vector is selected from the group consisting of an adenoviral vector, an adeno-associated virus vector, a hybrid adeno-associated virus vector, a lentivirus vector, a pseudo-typed lentivirus vector, a herpes simplex virus vector, a vaccinia virus vector, and a retroviral vector.

6. The method as claimed in claim 1, wherein said angiopoietin-1 molecule is an angiopoietin-1 polypeptide or a functional fragment thereof.

7. The method as claimed in claim 1, wherein said subject is a patient having a disorder, wherein said disorder is associated with an ischemic event.

8. The method as claimed in claim 7, wherein said ischemic event is selected from the group consisting of cerebrovascular ischemia, subarachnoid hemorrhage, myocardial infarct, surgery and trauma.

9. The method as claimed in claim 1, wherein said subject is a patient with diabetes.

10. The method as claimed in claim 9, wherein said patient with diabetes suffers from chronic non-healing diabetic wounds.

11. The method as claimed in claim 9, wherein said patient with diabetes suffers from diabetic neuropathy.

12. The method as claimed in claim 1, wherein said subject is a patient undergoing organ transplantation.

13. The method as claimed in claim 12, wherein said transplant is selected from the group consisting of an organ, a tissue, and a graft.

14. A method for recruiting endothelial precursor cells in a subject comprising administering a therapeutically effective amount of an angiopoietin-1 molecule to a site in said subject, wherein said site is substantially denuded of endothelial cells and said administration effects recruitment of endothelial precursor cells to said site.

15. The method of claim 1, further comprising administering therapeutically effective amounts of an insulin-like growth factor 1 molecule and a platelet derived growth factor B molecule which act synergistically to recruit endothelial precursor cells to said wound site.

16. The method of claim 15, further comprising the administration of IL-10.
